(19) United States
(12) Patent Application Publication
(10) Pub. No.: US 2011/0184310 A1
(43) Pub. Date: Jul. 28, 2011

(54) METHOD OF HEATING A SHAPE MEMORY ALLOY OF A SURGICAL INSTRUMENT

(76) Inventor: Joe Denton BROWN, Panama City, FL (US)

(21) Appl. No.: 13/012,931

(22) Filed: Jan. 25, 2011

Related U.S. Application Data
(60) Provisional application No. 61/298,709, filed on Jan. 27, 2010.

(51) Int. Cl. A61B 5/00; H05B 1/00

(52) U.S. Cl. 600/549; 219/162

(57) ABSTRACT

The shape of a surgical instrument is changed during a surgical procedure by using a laser to heat a shape memory alloy or other shape memory material in the surgical instrument, thereby causing the instrument to assume a predetermined shape, while at the same time monitoring the heating site to prevent overheating.

This application claims the benefit of provisional U.S. Patent Application Ser. No. 61/298,709, filed Jan. 27, 2010, and incorporated by reference herein.

### BACKGROUND OF THE INVENTION

1. Field of the Invention

This invention relates to an improvement to the methods described in the Inventor's U.S. Pat. Nos. 7,374,564 and 6,966,906, in which the shape of a surgical instrument is changed during a surgical procedure by heating the surgical instrument and thereby activating a shape memory alloy, or other shape memory material, in the instrument.

According to the methods described in U.S. Pat. Nos. 7,374,564 and 6,966,906, the surgical instrument is heated during a surgical or therapeutic procedure by delivering an irrigating fluid having an elevated temperature, and in particular by delivering to or bathing the instrument in a fluid having a temperature that is a few degrees above body temperature.

The present invention modifies this method by using a laser to heat the instrument. Control of the heating and/or prevention of overheating, is ensured by using feedback arrangements such as, by way of example and not limitation, the ones disclosed in the Inventor's U.S. Patent Publication Nos. 2007/0167937 (“Endovascular Method and Apparatus with Electrical Feedback”) and 2007/0049911 (“Endovascular Method and Apparatus with Feedback”), or the LaserGuard™ system sold by Optical Integrity, Inc.

2. Description of Related Art

The Inventor's U.S. Pat. Nos. 7,374,564 and 6,966,906 both describe surgical instruments that include a section made of a shape memory alloy such as Nitinol™. During a surgical operation, the shape memory alloy is heated to cause the shape memory alloy to change shape, thereby deflecting the instrument. For example, U.S. Pat. No. 6,966,906 discloses use of the shape memory alloy to deflect a laser fiber or endoscope, while U.S. Pat. No. 7,374,564 applies the shape memory alloy to a urological retrieval coil, also known as a stone cone. Other instruments to which shape memory alloys may be applied in order to cause a deflection or shape change when heated (or cooled) include heart valve clamps, guide wires, stents, sutures, and so forth.

The preferred heating method disclosed in U.S. Pat. Nos. 7,374,564 and 6,966,906 is to bathe the instrument in a fluid having a temperature that is above the transformation temperature of the shape memory alloy. In order to prevent injury to the patient, however, the temperature can only be a few degrees higher than body temperature.

Because precise control of the fluid temperature at the treatment site is difficult, the proposed method of heating the surgical instrument has the disadvantage that it may be difficult to achieve rapid changes in temperature and thereby control the timing of the transformation or shape-change. As a result, it is difficult to control the timing of the transformation or shape-change. Other heating methods proposed in the two patents, such as electrical heating, require additional apparatus and are difficult to implement, and carry the risk of overheating and injury to the patient.

A need therefore exists for a way to more precisely control heating of the shape memory alloy (or other shape memory material) of a surgical instrument in order to deflect or cause the instrument to change shape during a surgical procedure, and yet which is practical to implement and does not present a risk to the patient.

### SUMMARY OF THE INVENTION

It is accordingly an objective of the invention to provide an improved method of heating a shape memory alloy or other shape memory material of a surgical instrument, which is practical to implement and minimizes risk of injury to the patient.

This objective is achieved, in accordance with the principles of a preferred embodiment of the invention, by providing a method of heating a shape memory alloy or other shape memory material of a surgical instrument that includes the steps of:
directing a laser at the instrument in order to heat the shape memory alloy or other material to a transformation temperature and thereby cause the alloy to change shape; and
monitoring the heating site for signs of overheating using a feedback system and/or method that either detects a temperature of the instrument or of tissues surrounding the heating site, or radiation emitted by the instrument or tissues.

Monitoring of the heating site can be carried out in a variety of ways. Especially suitable systems and methods are disclosed in U.S. Patent Publication Nos. 2007/0167937 (“Endovascular Method and Apparatus with Electrical Feedback”) and 2007/0049911 (“Endovascular Method and Apparatus with Feedback”), as well as U.S. Patent Application Ser. No. 61/232,314, filed Aug. 7, 2009, and are embodied by the LaserGuard™ system sold by Optical Integrity, Inc. However, it will be appreciated that other monitoring systems and methods may also be utilized, and that the invention is not intended to be limited to the methods and systems specifically referred-to above.

The monitoring system and method may, for example, be arranged to detect radiation transmitted back through the laser delivery fiber or another optical fiber, or be responsive to a radiation or temperature sensor that communicates electrically or wirelessly with a monitoring device. In either case, the monitoring device should preferably be arranged to modulate or terminate delivery of laser energy to the surgical instrument upon detection of overheating, or to cause withdrawal or pullback of the laser delivery fiber. In addition, a sacrificial element, such as a coating or sheath, may be included on or near the surgical instrument in order to enhance the ability to detect overheating.

It should be appreciated that the term “surgical instrument” as used herein encompasses any device or manmade object or part that is inserted into a patient, including a laser delivery fiber and any devices or parts used to facilitate insertion of the fiber, such as an endoscope or introducer. It will be appreciated that the “overheating” detected by the method and apparatus of the invention may encompass overheating of tissues or any portion of a surgical instrument that, if undetected, could eventually result in injury to the patient or damage to the instrument. Further, it is to be understood that while particular surgical procedures may be mentioned herein, the invention is not to be limited to any particular surgical procedure, but rather should encompass any surgical procedure in which radiation is delivered to a treatment site by an instrument inserted into the patient to a position near the treatment site. These procedures include, but are not limited to, those involving urological stone removal or destruction, vascular treatment, and laser liposuction.

### BRIEF DESCRIPTION OF THE DRAWINGS

FIGS. 1 and 2 show a prior art surgical instrument to which the principles of the invention may be applied.

FIG. 3 is a schematic diagram of a laser delivery and monitoring system that may be utilized to implement the principles of a preferred embodiment of the invention.

FIG. 4 is a schematic diagram of an alternative laser delivery and monitoring system that may be utilized to implement the principles of the preferred embodiment.

FIG. 5 is a flowchart of a method of heating a shape memory alloy of a surgical instrument according to the preferred embodiment.

### DETAILED DESCRIPTION OF THE PREFERRED EMBODIMENTS

FIG. 1 shows one example of a surgical instrument 1 to which the principles of the invention may be applied. The surgical instrument 1 is a urological retrieval coil of the type disclosed in U.S. Pat. No. 7,374,564, which is made, at least in part, of a shape memory alloy or other shape material such as a shape memory polymer. The instrument 1 is inserted through a sheath 2 into a passage 3 such as the urinary tract of a patient suffering from a stone 4. As shown in FIG. 1, the passage is filled with an irrigating fluid 5 at a temperature T₁. The shape memory alloy has a transformation temperature T₂ such that the instrument maintains an uncoiled shape so long as the temperature of the irrigating fluid 5 is less than T₂, thereby allowing the instrument to be moved past the stone 4. However, when the temperature of the irrigating fluid exceeds T₂, the instrument assumes a predetermined coil shape, such that when the instrument is withdrawn toward the sheath 2, it captures the stone 4 to enable the stone to be removed from the patient.

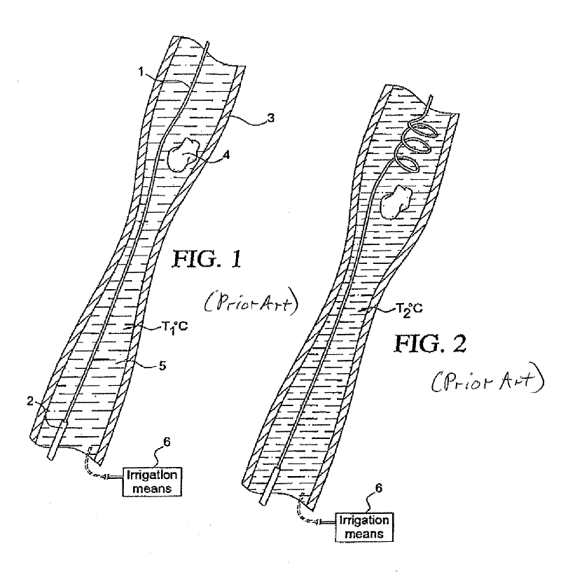

In the prior art shown in FIGS. 1 and 2, heating of the instrument is accomplished by raising the temperature of the irrigation fluid. However, there are many applications where this is impractical, or where more precise control of the transition is requirement. Accordingly, the present invention modifies the method shown in FIGS. 1 and 2 by instead heating the instrument with a laser.

The instrument to which the principles of the invention are applied may be a urological retrieval coil, such as the one illustrated in FIGS. 1 and 2, or any other surgical instrument that includes a shape memory alloy for the purpose of achieving a shape change at a predetermined temperature. The shape memory alloy may be Nitinol™ as disclosed in U.S. Pat. No. 7,374,564, or any other shape memory alloy or material having a transition temperature that makes it suitable for surgical applications.

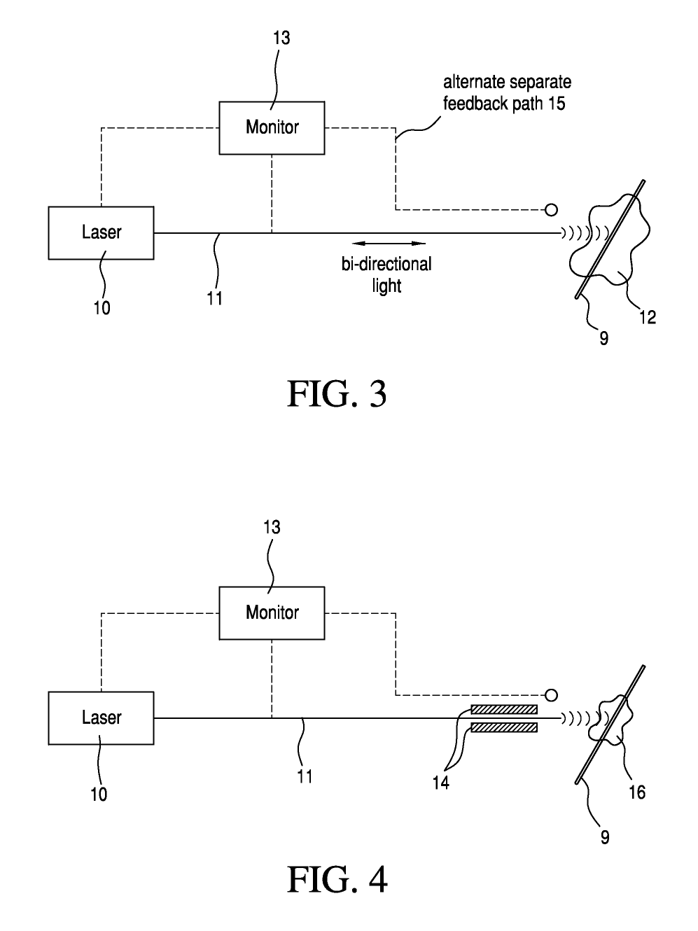

FIGS. 3 and 4 show implementation examples of the method of the invention, in which a laser is arranged to heat the instrument while being monitored by a suitable feedback arrangement. In the monitoring arrangement shown in FIG. 3, which generally corresponds to the system disclosed in U.S. Patent No. 61/232,314, filed Aug. 7, 2009, laser 10 is arranged to supply radiation to an optical fiber 11 for delivering energy to a surgical instrument 9. As the instrument is heated, a monitor 13 checks for radiation 12 emitted at the treatment site, which may be in the form of infrared radiation or, in the case of actual burning of tissues, visible light. The monitor 13 may be arranged to detect the radiation in a number of different ways, such as by detecting visible light transmitted back through the same fiber that supplies the laser energy to the treatment site, or by a separate detector and feedback path 15 having a sensor or detector for either radiation or temperature. When the monitor 13 detects that overheating has occurred or is imminent, an audible or visible alert is provided to an operator of the laser, and/or a feedback signal is supplied to the laser to either modulate or terminate the laser output. In the case of radiation feedback, the monitoring system may be arranged to detect not only the intensity of radiation, but also an amplitude profile or shape of the radiation signal over time to thereby distinguish between radiation resulting from heating of the surgical instrument to effect the shape change and radiation resulting from overheating of instruments or tissues.

In a modification of the system disclosed in FIG. 3, a sacrificial element 14 may be provided for absorbing selective radiation wavelengths 16. The sacrificial element 14 may be in the form of a coating on a surface exposed to the damage radiation, such as the working channel of an endoscope, may be integrated with or form a part of the surface to be protected, or may be a discrete element that can be disposed near a surface or tissue to be protected. Alternatively the sacrificial element 14 may take the form of a sheath into which the surgical instrument is inserted, and that is inserted together with the instrument into an endoscope. As the sacrificial element 14 absorbs selected wavelengths of the damage radiation 12, it emits additional radiation 13 and/or increases in temperature for detection using bi-directional light or a separate feedback path.

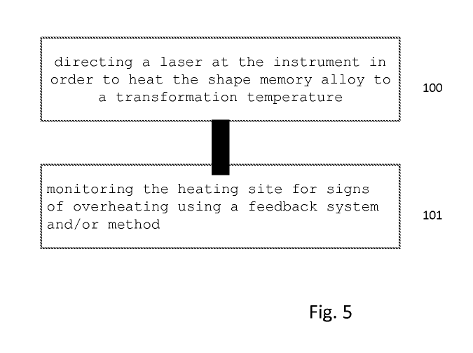

As illustrated in FIG. 5, the invention thus provides a method of heating a shape memory alloy for the purpose of causing a surgical instrument containing the alloy to change to a predetermined shape, for example to deflect the instrument or to form a coil, basket, stent, or other desired shape, the method including the steps of:
Step 100: directing a laser at the instrument in order to heat the shape memory alloy to a transformation temperature and thereby cause the alloy to change shape; and
Step 101: monitoring the heating site for signs of overheating using a feedback system and/or method that either detects a temperature of the instrument or of tissues surrounding the heating site, or radiation emitted by the instrument or tissues.
   It will of course be appreciated that step 100 is only carried out for so long as it is necessary for the instrument to maintain the shape that it assumes upon heating.

Having thus described various preferred embodiments of the invention in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with the appended claims.

I claim:

1. A method of heating a shape memory alloy or material of a surgical instrument during a surgical procedure, comprising the steps of:
while the surgical instrument is in a patient, directing a laser at the instrument in order to heat the shape memory alloy or material to a transformation temperature and thereby cause the alloy or material to change shape; and
monitoring a site at which the surgical instrument is being heated for signs that the instrument or tissues surrounding the instrument are being overheated.

2. A method as claimed in claim 1, wherein said site is monitored by detecting a temperature of the instrument.

3. A method as claimed in claim 1, wherein said site is monitored by detecting a temperature of tissues surrounding the heating site.

4. A method as claimed in claim 1, wherein said site is monitored by detecting radiation emitted by the instrument or tissues.

5. A method as claimed in claim 4, wherein said site is monitored by detecting said radiation upon transmission of the radiation back through the laser delivery fiber.

6. A method as claimed in claim 1, wherein said site is monitored by a temperature or radiation sensor at the treatment site.

7. A method as claimed in claim 1, wherein said monitoring step includes the step of monitoring an amplitude profile or shape of a temperature or radiation signal to distinguish between different heat or radiation sources at the treatment site.

8. A method as claimed in claim 1, wherein said instrument is an instrument used for urological stone removal or destruction.

9. A method as claimed in claim 1, wherein said instrument is an instrument used for vascular treatment.

10. A method as claimed in claim 1, wherein said instrument is an instrument used for laser liposuction.
